(19) United States
(12) Patent Application Publication
(10) Pub. No.: US 2002/0147210 A1
(43) Pub. Date: Oct. 10, 2002

(54) Methods for delaying recurrence of herpes virus symptoms

(75) Inventor: Michael H. Smith, St. Paul, MN (US)

Correspondence Address: 3M Innovative Properties Company, Office of Intellectual Property Counsel, PO Box 33427, St. Paul, MN, 55133-3427, US

(73) Assignee: 3M Innovative Properties Company

(21) Appl. No.: 10/108,961

(22) Filed: Mar. 28, 2002

Related U.S. Application Data
(63) Continuation of application No. 09/932,479, filed on Aug. 17, 2001.
(60) Provisional application No. 60/240,946, filed on Sep. 15, 2000.

(51) Int. Cl. A61K 31/4745

(52) U.S. Cl. 514/293

(57) ABSTRACT

Novel dosing regimens of resiquimod formulations are disclosed for delaying recurrence of herpetic lesions in patients affected with a herpes virus infection. Preferably, dosing regimens include administering a pharmaceutical formulation containing resiquimod to a herpetic lesion once a week for at least one week.

### FIELD OF THE INVENTION

The invention is directed to novel dosing regimens for the administration of resiquimod. In some embodiments, the invention is particularly advantageous for delaying recurrence of symptoms associated with infection by double-stranded DNA viruses such as herpes simplex virus types 1 (HSV-1) and 2 (HSV-2).

### BACKGROUND OF THE INVENTION

Approximately 600,00 new cases of herpes simplex virus are diagnosed annually in the United States. The total number of people infected in the United States is estimated to be more than 40 million.

Herpes simplex virus is composed of a double-stranded DNA nucleoprotein core surrounded by an icosahedral protein capsid, which in turn is enclosed in a lipid and glycoprotein outer envelope. It is a member of a family of eight known related human herpes viruses, including herpes simplex virus types 1 (HSV-1) and 2 (HSV-2), varicella-zoster virus (VZV), Epstein-Barr virus (EBV), cytomegalovirus (CMV), human herpes virus 6 (HHV-6), human herpes virus 7 (HHV-7) and human herpes virus 8 (HHV-8). Many herpes viruses are capable of establishing latency in certain cell types, resulting in persistent infection.

Herpes simplex lesions can generally occur as a result of a primary (initial) infection or as a result of a recurrence (reactivation) at the same site. During acute primary infection, the herpes simplex virus may establish latent infection in the nerve root ganglia that corresponds to the cutaneous or mucous membrane site of inoculation. Herpes simplex skin infections are usually located in the orolabial, genital or anorectal areas. Orolabial HSV infection is typically HSV-1 and genital is typically HSV-2; however, each site may be infected with the other HSV type. Following orolabial infection, HSV becomes latent in the trigeminal ganglia and after genital or anorectal infection, HSV becomes latent in the sacral ganglia. A variety of stimuli, such as ultraviolet light, fever, menstruation, stress, local skin trauma, or trauma to the sensory nerve, can reactivate latent HSV.

Most cases of HSV infection can be diagnosed by the morphological characteristics of the clinical symptoms including small, grouped vesicles on erythematous bases which then pustulate, ulcerate and later form a crust. Systemic symptoms can occur (e.g., fever, headache, myalgia, and malaise), but are more commonly associated with primary infection, especially genital herpes.

Resiquimod (4-amino-α,α-dimethyl-2-ethoxymethyl-1H-imidazo[4,5-c]quinoline-1-ethanol) is a member of the imidazoquinoline family of immune response modifiers. One member of this family, known as imiquimod, has been commercialized in a topical formulation, Aldara™, by 3M Company, St. Paul, Minn., for the treatment of anogenital warts associated with human papillomavirus. Resiquimod has demonstrated potent antiviral activity in animal models. This activity appears to be mediated predominantly through the induction of cytokines, including interferon-α (IFN-α) and interleukin 12 (IL-12). Resiquimod has been shown to be useful as a vaccine adjuvant. It has also been shown to enhance T helper type 1 cytokine release while suppressing T helper type 2 cytokine production.

Although some of the beneficial effects of resiquimod are known, the discovery of additional therapeutic or prophylactic benefits of this compound via novel dosing regimens is ongoing. The present disclosure is directed to such discoveries.

### SUMMARY OF THE INVENTION

The invention is directed to dosing regimens effective for delaying the recurrence of clinical symptoms associated with a human herpes virus infection.

It will be noted that in several places throughout the specification, guidance is provided through lists of examples. In each instance, the recited list serves only as a representative group. It is not meant, however, that the list is exclusive.

In one embodiment, the invention provides a method for delaying recurrence of symptoms caused by a herpes virus infection. The method includes a step of administering a pharmaceutical formulation containing from about 0.001 percent to about 0.05 percent by weight of resiquimod, based on total weight of the formulation, to a herpes virus lesion. The formulation can be administered to the lesion and can be administered until the lesion is resolved, or for a period of time beyond resolution of the lesion. The formulation can be administered at least one time per week, typically at least two times per week or three times per week, and in some embodiments, daily or every other day. The invention is particularly advantageous for use in delaying recurrence of symptoms associated with HSV-1 or HSV-2. In some embodiments, recurrence of clinical symptoms can be delayed for at least 120 days after first administration of the pharmaceutical formulation, typically for at least 120 days after the completion of one treatment cycle.

In another embodiment, the invention provides a method for delaying recurrence of a herpes virus infection including a step of topically administering a pharmaceutical formulation including 0.01 percent, based on total weight of the formulation, of resiquimod to a herpes virus lesion at least one time per week for at least one week.

### DETAILED DESCRIPTION

The present invention is directed to novel dosing regimens for delaying the recurrence of clinical symptoms associated with human herpes virus infection after resolution of prior clinical symptoms caused by the virus. As used herein, a “herpes virus” refers to members of the herpetoviridae family including herpes simplex virus types 1 (HSV-1) and 2 (HSV-2), varicella-zoster virus (VZV), Epstein-Barr virus (EBV), cytomegalovirus (CMV), human herpes virus 6 (HHV-6), human herpes virus 7 (HHV-7) and human herpes virus 8 (HHV-8). The invention is particularly advantageous for use to delay recrudescence of symptoms associated with HSV-1 and HSV-2.

Methods for preparing resiquimod are known and disclosed in, for example, U.S. Pat. No. 5,389,640 (Gerster). Pharmaceutical formulations containing resiquimod and methods for preparing them are disclosed in U.S. Pat. No. 5,939,090 (Beaurline). Other suitable formulations are known and can be used according to the invention including, for example, formulations disclosed in U.S. Pat. No. 6,245,776 (Skwierczynski). The entire disclosure of each of these patents are incorporated herein by reference.

Clinical symptoms associated with herpes viruses are well known. Lesions typical for orolabial herpes include painful vesicular eruptions with distinct vesicles appearing on the lips, tongue, or buccal mucosa. The vesicles can quickly coalesce and rupture to form shallow ulcers covered with whitish yellow necrotic material. Clinical symptoms typical for genital herpes include small, grouped vesicles on erythematous bases. The vesicles can unroof spontaneously or by direct abrasion to form, ulcerations. The ulcerations typically form a crust and then undergo re-epithelialization.

According to the invention, a pharmaceutical formulation containing resiquimod can be administered to the lesions when first apparent. The formulation may also be applied to the visible lesion site after resolution of the lesion. The pharmaceutical formulation can be topically applied to the lesions or lesion sites. The pharmaceutical formulation contains resiquimod in an amount about 0.001 to 0.05 percent by weight, preferably about 0.01 percent by weight, based on the total weight of the formulation.

The formulation can be administered to lesions at least one time per week (1×/week). Administration of the formulation is typically greater than one time per week (1×/week), more typically at least two times per week (2×/week) or three times per week (3×/week) and, in some embodiments, daily or every other day. Because treatment according to the invention is most easily administered to lesions, treatment typically commences once lesions are visible. Treatment according to the invention may be administered for a period of about 1 to 18 weeks, typically about 1 to 6 weeks, and preferably about 3 to 4 weeks. Generally about 125 mg ±10% of gel per 10 cm² of treatment area or about 150 mg ±10% of gel per 15 cm² of treatment area or about 225 mg ±10% of gel per 20 cm² of treatment area is applied. The pharmaceutical formulation can remain on the lesions for about 6 to 12 hours, typically about 8 to 10 hours.

In some embodiments, when a formulation containing resiquimod was administered to a population of patients having herpetic lesions, after cessation of treatment, clinical symptoms did not recur for a median time of at least 120 days, typically at least 150 days, in some embodiments at least 172 days and in some embodiments at least 190 days.

Unlike other anti-herpetic compounds which inhibit recurrence of herpes virus while the drug is being administered, the regimens disclosed herein provide for inhibition of recurrence of herpes virus symptoms after cessation of resiquimod administration. While not wishing to be bound by a single theory, it is believed that the advantageous features of the present invention may be due to the coupling of the immune enhancing cytokines induced by resiquimod with the endogenous antigen which is present during the recurrence to provide enhanced cell mediated immunity.

### EXAMPLES

The following Examples are provided to further describe the invention and should not be intended to limit the scope of the invention to the Examples.

### Example 1

Preparation of Resiquimod Formulations

Propylene glycol (700 g) and resiquimod (4-amino-2-ethoxymethyl-α,α-dimethyl-1H-imidazo[4,5-c]quinoline-1-ethanol, 1.4 g) were added to a 1000 mL glass beaker. The resulting mixture was heated (about 55° C.) with stirring until all of the resiquimod was dissolved. The resulting solution was added to the mixing bowl of a ROSS LDM-4 mixer. Triacetin (11,968.7 g) was added to the mixing bowl and the resulting mixture was mixed for 10 minutes at 36 rpm. Colloidal silicon dioxide (1,330.0 g, AEROSIL®200 from Degussa, Frankfurt, Germany) was added in five parts. After each addition the resulting mixture was mixed at ambient pressure for 1 to 2 minutes at 36 rpm and then under vacuum (18 inches of Hg below ambient pressure, about 4.0×10⁵ Pa) for about 9 minutes at 36 rpm. The sides of the mixing bowl and the mixing blades were scraped. The formulation was mixed under vacuum (17 inches of Hg below ambient pressure, about 4.3 ×10⁵ Pa) for about 10 minutes at 36 rpm. The resulting gel contained 0.01% resiquimod, 5.0% propylene glycol, 9.5% colloidal silicon dioxide, and 85.49% triacetin.

Using the method described above, a second formulation was prepared by combining 7.0 g of resiquimod, 700.0 g of propylene glycol, 11963.0 g of triacetin and 1,330.0 g of colloidal silicon dioxide. The resulting gel contained 0.05% resiquimod, 5.0% propylene glycol, 9.5% colloidal silicon dioxide, and 85.45% triacetin.

Clinical Study of Resiquimod Formulations applied to Herpetic Patients

A randomized, double-blind vehicle (placebo)-controlled study was performed to evaluate single and multi-weekly doses of the resiquimod formulations prepared above topically applied to herpetic lesions on the skin of the external genitalia.

52 otherwise healthy patients (20 to 60 years of age, inclusive) with a history of recurrent herpes genitalis (≧6 episodes per year) participated in the study. All patients had clinically inactive herpes genitalis two weeks before and at the time of screening for entry into the study. After passing screening, patients were enrolled into a 12-week eligibility period to qualify for treatment by virtue of experiencing a herpetic recurrence. During the eligibility period patients' qualified for the treatment period by coming to the study site for treatment visit one within 24 hours of a recurrence. At this visit patients were stratified by sex and enrolled sequentially into an active treatment or vehicle treatment regimen.

The treatment period began with the first treatment visit and ended with the final treatment visit. The treatment groups were 0.05 percent resiquimod containing formulation or formulation alone (vehicle) 1×/week for 4 weeks; 0.05 percent resiquimod containing formulation or formulation alone 2×/week for 3 weeks; 0.01 percent resiquimod formulation or formulation alone 2×/week for 3 weeks; or 0.01 percent resiquimod formulation or formulation alone 3×/week for 3 weeks.

Efficacy evaluations included assessment of time to recurrence in a 6 month observation period; total number of recurrences in the observation period; and the size, number and duration of lesions during recurrences in the observation period.

After the first visit, all patients returned the following day, and subsequently on their scheduled visits according to their dosing regimens, and 5 to 7 days after their last application of study formulation. The 6-month observation period began immediately after the treatment period, and patients returned to the clinic at 1, 3 and 6 months. During the observation period patients also returned to the clinic within 72 hours of each recurrence, and a clinic staff personnel verified and documented all herpetic lesions. The results of the study are shown in Table I below.

TABLE I

| | | | Median No. Days |
|---|---|---|---|
| Resiquimod | Dosing | Duration of | to Recurrence after |
| Concentration | Frequency | Treatment | treatment cessation |
| 0.05% | 1×/wk | 4 weeks | >60 |
| 0.05% | 2×/wk | 3 weeks | 105 |
| 0.01% | 2×/wk | 3 weeks | 172.5 |
| 0.01% | 3×/wk | 3 weeks | >195 |
| Vehicle* | | | 57 |

*Summary of the vehicle controls from the four treatment groups

Through this study, it was discovered that delay in recurrence of herpetic lesions was more prolonged using lower concentration resiquimod formulations than higher concentration formulations. The delay in recurrence is also associated with increasing frequency of administration rather than increasing concentration.

### Example 2

### Topical Application of IRM Formulation to Orolabial Herpetic Lesions

A pharmaceutical formulation containing 0.001 percent or 0.01 percent, by weight, based on total formulation weight, of resiquimod can be applied to orolabial lesions caused by a herpes virus. The pharmaceutical formulation can be topically applied to the lesions or lesion sites at least once per week for at least one a week using regimens and application methods as described herein.

From the foregoing detailed description and Examples, it will be evident that modifications and variations can be made in the products and processes of the invention without departing from the spirit or scope of the invention. Therefore, it is intended that all modifications and variations not departing from the spirit of the invention come within the scope of the claims and their equivalents.

We claim:

1. A method for delaying recrudescence of a herpes virus infection, the method comprising a step of topically administering a pharmaceutical formulation including from about 0.001 percent to about 0.05 percent by weight, based on total weight of the formulation, of 4-amino-α,α-dimethyl-2-ethoxymethyl-1H-imidazo[4,5-c]quinoline-1-ethanol to a herpes virus lesion at least one time per week for at least one week.

2. The method according to claim 1 wherein the 4-amino-α,α-dimethyl-2-ethoxymethyl-1H-imidazo[4,5-c]quinoline-1-ethanol is present in an amount of about 0.01 percent to about 0.05 percent by weight, based on total weight of the formulation.

3. The method according to claim 1 wherein the 4-amino-α,α-dimethyl-2-ethoxymethyl-1H-imidazo[4,5-c]quinoline-1-ethanol is present in an amount of about 0.01 percent by weight, based on total weight of the formulation.

4. The method according to claim 1 wherein the herpes virus lesion is an anogenital lesion.

5. The method according to claim 1 wherein the herpes virus lesion is an orolabial lesion.

6. The method according to claim 1 wherein the pharmaceutical formulation is administered at least one time per week for at least 2 weeks.

7. The method according to claim 1 wherein the pharmaceutical formulation is administered at least one time per week for at least 3 weeks.

8. The method according to claim 1 wherein the herpes virus infection is caused by HSV-2.

9. The method according to claim 1 wherein the herpes virus infection is caused by HSV-1.

10. The method according to claim 1 wherein the pharmaceutical formulation is administered at least two times per week.

11. The method according to claim 10 wherein the pharmaceutical formulation is administered for at least two weeks.

12. The method according to claim 10 wherein the pharmaceutical formulation is administered for at least three weeks.

13. The method according to claim 1 wherein the pharmaceutical formulation is administered at least three times per week.

14. The method according to claim 13 wherein the pharmaceutical formulation is administered for at least two weeks.

15. The method according to claim 13 wherein the pharmaceutical formulation is administered for at least three weeks.

16 The method according to claim 1 wherein the pharmaceutical formulation is administered every other day.

17. The method according to claim 16 wherein the pharmaceutical formulation is administered for at least two weeks.

18. The method according to claim 16 wherein the pharmaceutical formulation is administered for at least three weeks.

19. The method according to claim 1 wherein the pharmaceutical formulation is administered daily.

20. The method according to claim 19 wherein the pharmaceutical formulation is administered for at least two weeks.

21. The method according to claim 19 wherein the pharmaceutical formulation is administered for at least three weeks.

22. A method for delaying recurrence of clinical symptoms associated with a herpes virus infection in a patient, the method comprising a step of administering a pharmaceutical formulation including 0.01 percent of 4-amino-α,α dimethyl-2-ethoxymethyl-1H-imidazo[4,5-c]quinoline-1-ethanol, based on total weight of the formulation, to a lesion on the patient caused by the herpes virus at least until the lesion is resolved.

23. The method according to claim 22 wherein the pharmaceutical formulation is administered for a period of about 1 to 4 weeks after the lesion is resolved.

24. The method according to claim 22 wherein the pharmaceutical formulation is administered to the lesion at least one time per week.

25. The method according to claim 22 wherein the pharmaceutical formulation is administered to the lesion at least two times per week.

26. The method according to claim 22 wherein the pharmaceutical formulation is administered to the lesion at least three times per week.

27. The method according to claim 22 wherein the herpes virus lesion is an anogenital lesion.

28. The method according to claim 22 wherein the herpes virus lesion is an orolabial lesion.

29. The method according to claim 22 wherein the pharmaceutical formulation is topically administered to the lesion.

30. The method according to claim 22 wherein the herpes virus is HSV-2.

31. The method according to claim 22 wherein the herpes virus is HSV-1.

32. The method according to claim 22 wherein recurrence of clinical symptoms is delayed for at least 120 days after first administration of the pharmaceutical formulation to the lesion.
